(19) United States
(12) Patent Application Publication
(10) Pub. No.: US 2026/0077157 A1
(43) Pub. Date: Mar. 19, 2026

(54) URETHRAL CATHETER CAPABLE OF SMOOTHLY PASSING THROUGH URETHRAL STENOSIS SECTION

(71) Applicant: WUHAN XUNSHU TECHNOLOGY CO., LTD., Wuhan (CN)

(72) Inventors: Guxi XIANG, Wuhan (CN); Zhiwei OUYANG, Wuhan (CN)

(73) Assignee: WUHAN XUNSHU TECHNOLOGY CO., LTD., Wuhan (CN)

(21) Appl. No.: 19/398,119

(22) Filed: Nov. 24, 2025

Related U.S. Application Data
(63) Continuation-in-part of application No. 17/620,177, filed on Dec. 17, 2021.

(51) Int. Cl. A61M 25/00

(52) U.S. Cl. CPC A61M 25/0017; A61M 25/0041; A61M 25/007; A61M 25/0074; A61M 25/008; A61M 2025/0073; A61M 2202/0007; A61M 2202/0014; A61M 2202/0496; A61M 2206/10; A61M 2210/1089

(57) ABSTRACT

A catheter capable of smoothly passing through urethral stenosis section, within a head end of the catheter, there is provided with a cavity for forming a liquid turbulence therein; an inner surface of the cavity is separated apart from an outer surface of the check means with a gap therebetween to movably accommodate a guiding core head end of a guiding core; the guiding core head end plays a check role and the guiding core tube plays a guiding role; a lateral liquid channel is arranged between the front end of the guiding core and the head end of the catheter; the catheter body is more flexible than the head end of the catheter. During a catheter is being inserted into the urethra, the inventive catheter can reduce an injected liquid's impacting on a patient's urethra from the lateral liquid channel, and make the patient comfortable.

### CROSS-REFERENCE TO RELATED APPLICATIONS

This application is a continuation-in-part (CIP) application, with the US filing date of Dec. 17, 2021, and based on U.S. application Ser. No. 17/620,177 titled “Urethral Catheter Capable of Smoothly Passing Through Urethral Stenosis Section” in the name of Wuhan Xunshu Technology Co., Ltd.

### FIELD OF THE INVENTION

The present invention relates to a catheter, in particular to a catheter which can be easily inputted into a urethra of patients, especially can be operated to smoothly pass through the narrowest section of the urethra of patients, who suffered from stricture of prostate, or prostatic hyperplasia, etc.

### PRIOR ART

In prior art, in order to prevent a guiding core from penetrating out of a tube wall of a catheter, a complex structure is arranged. For example, a size of a hole for draining urine is reduced to be less than a diameter of the guiding core; a front end of the catheter is made to have a section of rigid cylinder wall; or a limiting means is set at a front section of the guiding core, etc., so as to prevent the guiding core from penetrating through any hole for discharging urine.

Those technical solutions in the prior art have the same problems as each other, i.e., the cross-sectional area for discharging urine within a catheter tube has to become smaller; a catheter's capacity for discharging urine, i.e. a maximum volume of urine discharged per minute, has to be reduced; a resistance for injecting a lubricant is increased; and any patient to use the catheter is far from being comfortable.

### SUMMARY OF THE INVENTION

The object of the invention is to provide a catheter, which is strong for discharging urine, is able to discharge a large volume of urine, has a low resistance to inject lubricant into the catheter, and makes any patient for using the catheter comfortable.

Thus, according to one aspect of the invention, there is provided a catheter, wherein a head end of the catheter is provided with a cavity for making liquid turbulence, an internal span of the cavity is greater than a mouth span thereof, and the cavity accommodates a check means located at a front end of a guiding core, the check means is a part of the guiding core that can be inserted into the guiding core tube from a tail end thereof and move forward within the guiding core tube; after moving out of the guiding core tube, the check means of the guiding core can expand outward, and a span of the expanded check means is greater than an opening diameter, i.e. the moth span, of the cavity at the head end of the catheter, a liquid channel is arranged between the front end of the guiding core and the head end of the catheter.

Preferably, the cavity comprises one or more cavities which are provided in series.

According to the second aspect of the invention, there is provided a catheter, wherein a clamping means is provided at the front end of the guiding core to match with a head end of the catheter; the head end of the catheter is provided with a cavity for making liquid turbulence.

According to a third aspect of the invention, there is provided a catheter, wherein an internal cross-sectional area at a front end of the catheter for discharging urine can be fully used up to as large as an inner hole area of the catheter; a head end of the catheter is provided with a cavity for making liquid turbulence.

According to a fourth aspect of the invention, there is provided a catheter, wherein a head end of the catheter is directly connected with a guiding core; the head end of the catheter is provided with a cavity for making liquid turbulence.

Preferably, a front end of the guiding core is directly connected with the head end of the catheter.

Preferably, a front end of the guiding core is matched with the head end of the catheter.

Preferably, the check means is arranged directly within the head end of the catheter.

Preferably, a front end of the guiding core is matched with a protrusion of a head end of the catheter.

Preferably, the clamping means at the front end of the guiding core is matched with the head end of the catheter.

Preferably, a liquid channel is arranged between the front end of the guiding core and the head end of the catheter.

Preferably, the head end of the catheter is provided with the cavity, and an internal span of the cavity is greater than a mouth span.

Preferably, the cavity comprises one or more cavities which are arranged in series.

Preferably, the cavity accommodates a check means located at the front end of the guiding core.

Preferably, the check means is arranged at a tip of the guiding core that can retract into the guiding core tube and move along the guiding core tube.

Preferably, the guiding core can expand outward after moving out of the guiding core tube.

Preferably, the span of the expanded tip portion of the guiding core is greater than an opening diameter of the cavity arranged at the head end of the catheter.

Preferably, a limiting means is arranged at a tail end of the guiding core and/or the guiding core tube so as to regulate a position of the guiding core within the guiding core tube, and regulate a size of the liquid channel.

Preferably, a hardness of the head end is greater than that of the catheter body.

Preferably, the tail end of the catheter is provided with a connecting means, which is provided with a conical discharging hole and a lubricating fluid injecting hole.

Preferably, the connecting means is provided with an extra urine discharging hole, or the connecting means is provided with a channel leading to the urine discharging hole.

According to the invention, the head end of the catheter is directly connected with the guiding core, there is no longer any inner flange within the catheter at the guiding head thereof as provided in prior art, and there is no extra part arranged inside the front end of the catheter, that is, there is no need to add any accessory means within the catheter tube as does the traditional catheter. Therefore, it is no longer necessary to have to occupy some internal cross-sectional area of the catheter, so it is no longer to have to sacrifice some volume for discharging urine per minute from the catheter. Moreover, a resistance for injecting lubricant is much less than that of the prior art.

According to the invention, differing from the prior art, a cavity for making liquid turbulence is independently arranged within the head end of the catheter; or a cavity for making liquid turbulence is formed by combining upper and lower parts of the head end of the catheter; or a cavity for making liquid turbulence is formed between a flange of the head end of the catheter and the inner wall of the catheter; the cavity can be composed of one or more cavities provided in series. The internal span of the cavity is greater than or equal to the span of the mouth (preferably greater than). The cavity can accommodate a check means or a clamping means. The check means is a head of the guiding core, the head can be expanded after entering the cavity. The clamping device is a portion of the guiding core, preventing the guiding core from withdrawing by clamping a hanging down column of the head end of the catheter after the head end of the guiding core enters the cavity.

According to the invention, there is provided the cavity which is not arranged in the head end of any traditional catheter, the cavity accommodates the check means or the clamping means, limiting any retraction of the guiding core within the guiding core tube in the catheter, so as to avoid any possibly damaging urethra and prevent from any risk that the guiding core would penetrate through the urine discharging hole of the catheter. Since an inventive cavity is provided to locate in front of any urine discharging hole in view of a longitudinal direction of the catheter from its tail end to its head end, a urine discharging efficiency of the catheter is not sacrificed, and a cross-sectional area for injecting lubricant into the catheter is fully used to increase up to a whole inner cross section.

On the contrary, in prior art, an auxiliary structure is added within the catheter so as to prevent the guiding core from accidentally passing through the urine discharging hole of catheter, so that the added auxiliary structure makes it need a greater force to inject lubricant into the catheter, and decreases an efficiency for discharging the urine. In particular, in prior art, in order to insert the catheter into urethra of patients with urethral stricture or benign prostatic hyperplasia, a guiding wire is placed within the catheter to assist inserting the catheter into the urethra. However, clinically, the guiding wire needs to move forward or retract within the catheter. During movement within the catheter, the guiding wire might accidently pass through a urine discharging hole of the catheter, damaging or even piercing the patient's urethra.

According to the invention, any retracting or forwarding displacement of the guiding core within the guiding core tube of the catheter is avoided, so that the guiding core are prevented from accidently penetrating through the urine discharge hole; at the same time, the lubricant injection can have a high efficiency, expanding the urethra, reducing a friction between a tube of the catheter and any patient's urethra, thus reducing pain of patients.

According to the invention, the front end of the guiding core is directly connected with the head end of the catheter, or the check means is directly installed on the tip of the catheter, so that the catheter is operated with an accurate guidance and an accurate lubrication during inserting into the urethra. As a result, the patients become comfortable when using the inventive catheter.

### BRIEF DESCRIPTION OF DRAWINGS

FIG. 1 is a schematic view of a catheter that can smoothly pass through a urethral stricture section according to the present invention.

FIG. 2 is a schematic view of one embodiment of a head end of a guiding core according to the present invention.

FIG. 3 is a schematic view of another embodiment of the head end of the guiding core according to the present invention.

FIG. 4 is a schematic view of another embodiment of the head end of the guiding core according to the present invention.

FIG. 5 is a schematic view of another embodiment of the head end of the guiding core according to the present invention.

### BEST MODE FOR CARRYING OUT THE INVENTION

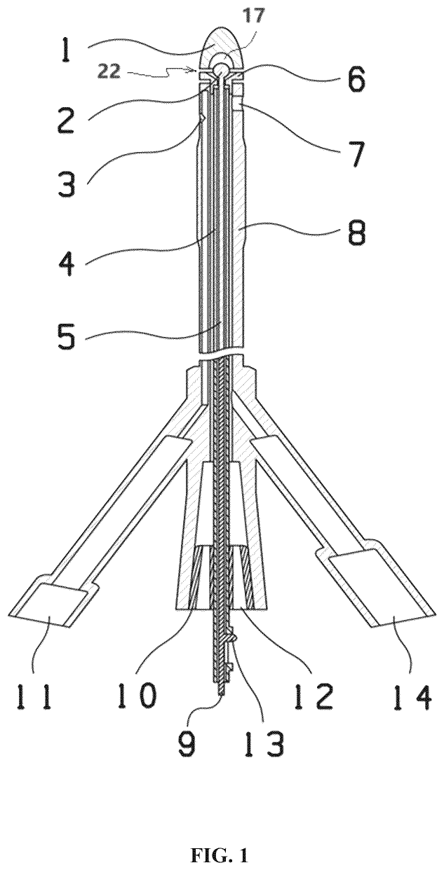

As shown in FIG. 1, according to an embodiment of the invention, an inventive catheter is a thin flexible tube which can be inserted into a human body, even smoothly passing through the narrowest section of urethra of the patient, so as to permit introduction of lubricant, or withdrawal of urine, or to keep a urine passageway open. The catheter includes: a head end upper part 1, a head end lower part 6, a catheter body 8, a guiding core head end 2, a guiding core tube 4, a guiding core 5, a liquid medicine inlet 11, a liquid medicine outlet 3, a conical urine draining hole 12 or a lubricant inlet 12, a flushing port 14 or a lubricant inlet 14, a urine draining hole 7 or a lubricant outlet 7, a connecting means 10, a guiding core pushing end 9, and a guiding core stopper 13.

In one embodiment, the guiding core head end 2 is also called a check means, which is located at top of the guiding core 5, and can move back and forth in a longitudinal direction within the guiding core tube 4. When the guiding core 5 slides into a cavity 17 arranged larger inside but smaller outside and located in the upper part 1 of the head end 2 of the catheter, the guiding core head end 2 performs its check function to restrict any backward movement of the guiding core 5 within the guiding core tube 4, so that the guiding core 5 is prevented from moving backward within the guiding core tube 4, and then prevented from accidently passing through the urine discharging hole 7 or the liquid medicine outlet 3 on a side wall of the catheter body 8.

The guiding core stopper 13 is a limiting means of the guiding core 5, which limits any excessive propulsion of the guiding core pushing end 9 of the guiding core 5, and regulates a size of the liquid channel 22.

The connecting means 10 is arranged at a rear end of the catheter body 8 to fit between an inner wall of the catheter body 8 and an outer wall of the guiding core 5, so that the guiding core 5 can be centered inside the catheter body 8. At the same time, the connecting means 10 is also provided with an interface for connecting with the conical urine discharging port 12 at tail end of the catheter and an interface for connecting with a lubricant injecting device. The connecting means 10 is a pushing and locking means for the guiding core 5, and also a fixing means for the catheter tube 8. The connecting means 10 is also a means for guiding and controlling any forward and backward movement of the guiding core 5 within the guiding core tube 4. The connecting means 10 is provided with the conical urine discharging hole 12 and a urethra flushing hole 14. Alternatively, the conical urine draining hole 12 and the urethra flushing hole 14 can also be used as lubricant injecting holes. The connecting means 10 has a lubricant channel, which can be butted with any lubricant injecting device.

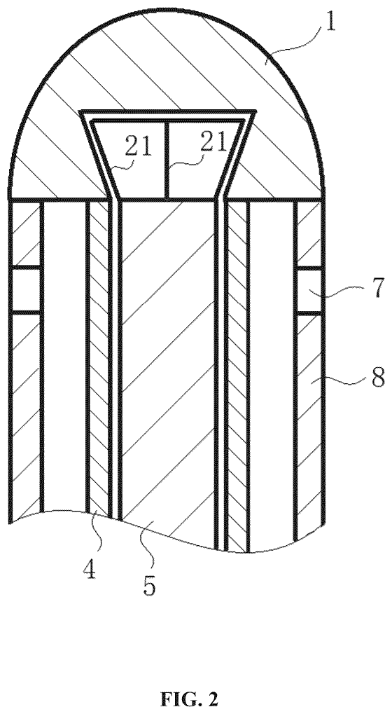

As shown in FIG. 2, in one embodiment, the check means, i.e., the guiding core head end 2, can be in a shape of a plurality of bones of an umbrella, can be folded or opened, can adopt multiple umbrella bones 21, or can be formed by bending an elastic metal wire 21 for many times. After the guiding core head end 2 of the guiding core 5 spreads out, the guiding core 5 remains within the guiding core tube 4. At this time, the guiding core head end 2 plays a check role, and the guiding core tube 4 plays a guiding role.

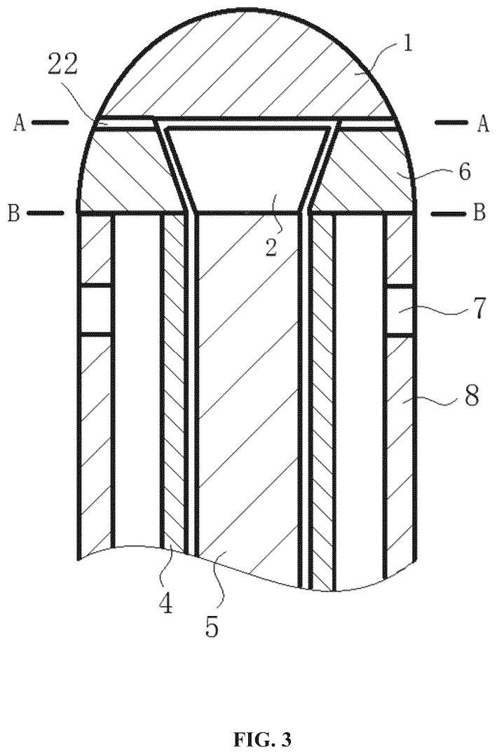

In the embodiment shown in FIG. 2, no small hole is provided in the head end of the catheter. In the embodiment shown in FIG. 3, one or more small holes 22 can also be arranged in the head end 2 of the catheter, so as to increase an efficiency for discharging urine.

In the embodiment shown in FIG. 3, the head end 2 of the catheter includes a head end upper part 1 and a head end lower part 6, and the head end upper part 1 and the head end lower part 6 can be bonded, inserted or clamped together. The small hole 22 is formed between the upper part 1 of the head end and the lower part 6 of the head end (FIG. 5), for discharging urine from a urethra or injecting a lubricant into the urethra. The lower part 6 of the head end is provided with a funnel-shaped space, and the guiding core head end 2 is matched in the funnel-shaped space in order to form a check means.

The head end 2 of the catheter can be bonded to the end of the catheter body 8, or bonded to the end of the guiding core tube 4 at the same time. For measuring a bonding strength, a separating force is tested at the end B of the catheter body 8 and at the position A where the small hole 22 is set at the head end 2 of the catheter. According to a technical standard of the invention, when an outer diameter of the catheter is less than 3.3 mm, the separating force is more than 0.8 kg; while when the outer diameter of the catheter is ≥3.3mm, the separating force is >1.1kg.

According to the invention, any lubricant can be injected into a gap between the outer wall of the guiding core tube 4 and the guiding core 5, or another gap between the guiding core tube 4 and the inner wall of the catheter body 8, and can be forced to discharge from the urine discharging hole 7 and/or the small hole 22 at the guiding core head end 2, so as to be pushed to enter the urethra. The lubricant can also be injected from inside of the guiding core tube 4, and the lubricant can be pushed to flow out from a side hole and/or top of the front end of the guiding core tube 4 into the urethra (FIG. 5). The lubricant can be injected through the liquid medicine injecting port 11, the urethra flushing port 14, or the conical urine discharging hole 12.

For a certain type of the catheter, a maximum diameter of the upper half 1 of the head end, a diameter of the lower half 6 of the head end, and a diameter of the catheter body 8 are the same as each other, preferably between 2-10 mm, and a thickness of the upper half 1 of the head end is not less than 0.1 mm. A bonding area between the lower half 6 of the head end and the end of the catheter body 8 is not less than a cross-sectional area of a tube wall of the catheter body 8.

According to the invention, requirements for a minimum separating force (kg) between the head end upper half 1 and the lower half 6 and a minimum separating force (kg) between the head end lower half 6 and the catheter body 8 are as follows: when an outer diameter of the catheter body 8 is 2 mm or 2.7 mm, the minimum separating force (kg)>0.7, preferably >0.8; When the outer diameter of the catheter body 8 is 2.7 mm, 3.3 mm, 4 mm, 4.7 mm, 5.3 mm, 6 mm, 6.7 mm, 7.3 mm, 8 mm, 8.7 mm, 9.3 mm or 10 mm, the minimum separating force (kg)>1, preferably >1.1.

According to the invention, a catheter product can be used anytime within 60 months or more.

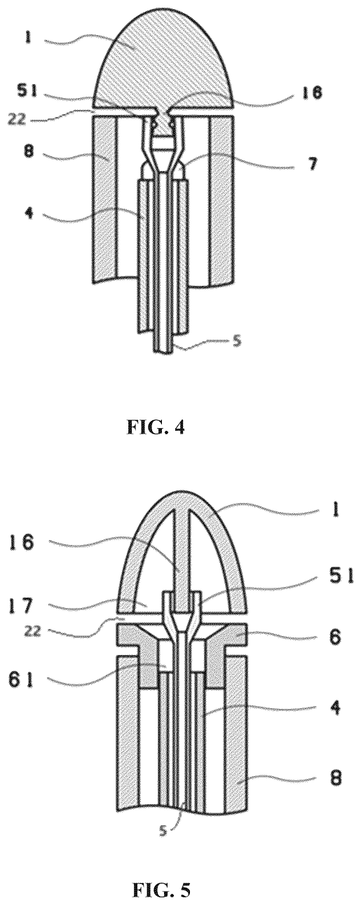

According to another embodiment of the invention, as shown in FIG. 4, the catheter head end 1 has a hanging down column 18 on the catheter head end 1, and the hanging down column 18 is integrated with the catheter head end 1, or installed on the catheter head end 1. There is a space 17 between the hanging down column 18 of the catheter head end 1 and the inner wall of the catheter body 8. A clamping means 51 arranged at the catheter head end 1 is inserted into the space 17 to clamp the hanging down column 18 of the catheter head end 1.

According to another embodiment of the invention, as shown in FIG. 5, the catheter head is divided into an upper part 1 and a lower part 6. The upper part 1 has a hanging down column 16 of the catheter head. The hanging down column 16 of the catheter head is an integral structure with the upper part 1 of the catheter head, or is installed on the upper part 1 of the catheter head. A draining hole 22 is arranged between the upper part 1 and the lower part 6. The lower part 6 is bonded to an end face of the catheter body 8. The lower part 6 has a central hole 61, and a clamping means 51 of the guiding core head end 2 of the guiding core 5 is inserted through the central hole 61 to clamp the hanging down column 16 of the catheter head. There may be a gap 22 between the lower part 6 and the guiding core 5 to allow the liquid to pass through. The lower part 6 and the guide core 5 can also be sealed. The lower part 6 includes a flange portion and a central tubular portion, the flange portion is bonded with an end face of the guiding core 5, while the central tubular portion is inserted into the guiding core tube 4 and bonded with the inner wall of the guiding core tube 4. In the upper part 1 of the catheter head, a space 17 is arranged around the hanging down column 16 of the catheter head so as to form a liquid turbulence, reduce the injected liquid impacting on the patient's urethra, and make the patient more comfortable.

A catheter according to the invention is characterized in that a head end 1 of the catheter is provided with a cavity 17, an internal span of the cavity is greater than its mouth span, and the cavity accommodates a check means 2 located at a front end of a guiding core 5.

The catheter according to the invention is characterized in that the head end 1, 6 of the catheter is directly connected with the guiding core 5.

The catheter according to the invention is characterized in that an internal cross-sectional area at a front end of the catheter for discharging urine is the same as a whole inner hole area of the catheter.

Preferably, the front end of the guiding core 5 is directly connected with the head end of the catheter.

Preferably, the front end of the guiding core 5 is matched with the head end of the catheter.

Preferably, the check means 2 is arranged directly within the head end of the catheter.

Preferably, the front end of the guiding core 5 is matched with a protrusion 16, 18 of the head end of the catheter.

Preferably, the clamping means 51 at the front end of the guiding core 5 is matched with the head end of the catheter.

Preferably, a liquid channel 22 is arranged between the front end of the guiding core 5 and the head end 1, 6 of the catheter.

Preferably, the head end 1, 6 of the catheter is provided with the cavity 17, and the internal span of the cavity 17 is greater than a mouth span thereof.

Preferably, the cavity 17 comprises one or more cavities which are arranged in series.

Preferably, the cavity 17 accommodates the check means 2 located at the front end of the guiding core 5.

Preferably, the check means 2 is a portion of the guiding core 5 that can shrink into the guiding core tube 4 and move within the guiding core tube 4.

Preferably, a head portion of the guiding core 5 can expand outward after moving out of the guiding core tube 4.

Preferably, a hardness of the head end 1, 6 is greater than that of the catheter body 8.

Preferably, a span of an expanded top guiding core 5 is greater than an opening outer diameter of the cavity 17 at the head end 1, 6 of the catheter.

Preferably, a limiting means 13 is arranged at a tail end of the guiding core 5 and/or the guiding core tube 4 so as to regulate a position of the guiding core 5 within the guiding core tube 4, so regulate a size of the liquid channel 22.

Preferably, the tail end of the catheter is provided with a connecting means 10, which is provided with a conical urine discharging hole 12 or a lubricant fluid injecting hole 12.

Preferably, the connecting means 10 is provided with a urine discharging hole 12, or the connecting means 10 is provided with a channel leading to the urine discharging hole 12.

According to the invention, any extending portion of a guiding head, inserted into the catheter in the prior art, is eliminated, and any parts arranged in the front end of the catheter are avoided. There are no additional attachments provided on an inner surface of a tube wall of the catheter with respect to the prior art, so that an internal cross-sectional area of the catheter for discharging urine becomes the same as an inner hole area of the catheter, thereby greatly increasing the urine discharging volume of the catheter. Moreover, an injection resistance of lubricant is also greatly reduced.

According to the present invention, there is provided a catheter capable of smoothly passing through urethral stenosis section so as to permit introduction of lubricant, withdrawal of urine, or keep a urine passageway open, wherein
within a head end 1, 6 of the catheter, there is provided with a cavity 17 for forming a liquid turbulence therein; a span of the cavity 17 within an internal space thereof is greater than that of a mouth of the cavity 17; a volume of the cavity 17 is larger than a volume of a check means 2; an inner surface of the cavity 17 is separated apart from an outer surface of the check means 2 with a gap therebetween to movably accommodate the check means 2 which is a guiding core head end 2 of a guiding core 5 and is arranged at a front end of the guiding core 5 so as to prevent the guiding core 5 from penetrating through a side wall hole 7 for discharging urine on a side wall of a catheter body 8, but the check means 2 is movably located at top of the guiding core 5 being able to move back and forth in a longitudinal direction within a guiding core tube 4, during which the guiding core head end 2 plays a check role and the guiding core tube 4 plays a guiding role; and the guiding core 5 is centered inside the catheter body 8;
a lateral liquid channel 22 is arranged between the front end of the guiding core 5 and the head end 1, 6 of the catheter so as to form the liquid turbulence within the cavity 17, reduce an injected liquid impacting on a patient's urethra from the lateral liquid channel 22, and make the patient comfortable; and
the catheter body 8 is more flexible than the head end 1, 6 of the catheter.

Preferably, the cavity 17 comprises one or, more cavities which are provided in series.

Preferably, the head end 1, 6 of the catheter is directly connected with the guiding core 5, and an internal cross-section area of a front end of the catheter is the same as an inner hole area of the catheter.

Preferably, the check means 2 or the guiding core head end 2 is directly arranged within the cavity 17 of the catheter and the front end of the guiding core 5 is in engagement with a hanging down column 16, 18 of the head end 1, 6 of the catheter.

Preferably, at a tail end of the guiding core 5 and/or a guiding core tube 4, there is provided with a limiting means 13, so as to regulate a position of the guiding core 5 within the guiding core tube 4, so regulate a size of the lateral liquid channel.

Preferably, at a tail end of the catheter, there is provided with a connecting means 10, and the connecting means 10 is provided with a conical hole 12 for discharging urine or for injecting lubrication.

Preferably, the front end of the guiding core 5 is integrated with the head end 1, 6 of the catheter; or the front end of the guiding core 5 is matched with the head end 1, 6 of the catheter; or the check means 2, 51 or the guiding core head end 2 is directly arranged within the head end 1, 6 of the catheter.

Preferably, a clamping means 51 of the front end of the guiding core 5 is matched with the head end 1, 6 of the catheter.

Preferably, in view of a longitudinal direction of the catheter from its tail end to its head end 1, 6, the cavity 17 is provided to locate in front of any hole for discharging urine, making a cross-sectional area for injecting lubricant into the catheter is fully used to increase up to a whole inner cross section.

Preferably, the cavity 17 is arranged to communicate with the lateral liquid channel 22, so as to form the liquid turbulence in a space of the cavity 17 before a liquid is injected out of the head end 1, 6.

Preferably, a lubricant is injected through one of at least three ports 11, 12, 14 for injecting lubricant; while urine is discharged from one of at least two channels 12, 14 for discharging urine.

Preferably, the head end 2 is operated to shrink into a guiding core tube 4 and move forward within the guiding core tube 4; after moving out of the guiding core tube 4, the head end 2 of the guiding core 5 expands outward, and the span of the expanded head end 2 of the guiding core 5 is greater than a size of the mouth of the cavity 17 at the head end 2 of the catheter.

Preferably, the lateral liquid channel 22 communicates with a cylinder-shaped space between an outer cylinder surface of the guiding core 5 and an inner wall of the guiding core tube 4, and/or between an outer surface of the guiding core tube 4 and an inner wall of the catheter.

Preferably, the connecting means 10 is further provided with a secondary hole for discharging urine, or the connecting means 10 is provided with a channel communicating with the hole 12 for discharging urine.

Preferably, any accessory means within the guiding core tube 4 of the catheter is excluded so as to prevent from occupying any internal cross-sectional area of the catheter, prevent from sacrificing some volume for discharging urine by the catheter per minute; and prevent from increasing a resistance for injecting lubricant, while making a high efficiency for injecting lubricant, expanding a patient's urethra, reducing a friction between the catheter and the urethra, thus reducing any pain of the patient.

Preferably, any over retracting displacement of the guiding core 5 within the guiding core tube 4 of the catheter is avoided, so that the guiding core 5 is prevented from accidently penetrating through a front hole 7 for discharge urine.

Preferably, within the head end 2 of the catheter, there is provided a space 17 to communicate with the lateral liquid channel 22, so as to form the liquid turbulence in the space 17 before any liquid is injected out of the head end 2 of the catheter.

Preferably, any additional attachment is prevented from providing on an inner surface of a tube wall of the catheter, so that in the catheter, an internal cross-sectional area for discharging urine becomes the same as the inner hole area of the catheter.

Several embodiments of the present invention are disclosed above. According to the above disclosure, those skilled in the art can make various changes, modifications, improvements, or developments to those discussed embodiments. However, all of above activities will fall within a scope of protection as claimed in those appended claims.

What is claimed is:

1. A catheter capable of smoothly passing through urethral stenosis section so as to permit introduction of lubricant, withdrawal of urine, or keep a urine passageway open, wherein
within a head end (1, 6) of the catheter, there is provided with a cavity (17) for forming a liquid turbulence therein; a span of the cavity (17) within an internal space thereof is greater than that of a mouth of the cavity (17); a volume of the cavity (17) is larger than a volume of a check means (2); an inner surface of the cavity (17) is separated apart from an outer surface of the check means (2) with a gap therebetween to movably accommodate the check means (2) which is a guiding core head end (2) of a guiding core (5) and is arranged at a front end of the guiding core (5) so as to prevent the guiding core (5) from penetrating through a side wall hole (7) for discharging urine on a side wall of a catheter body (8), but the check means (2) is movably located at top of the guiding core (5) being able to move back and forth in a longitudinal direction within a guiding core tube (4), during which the guiding core head end (2) plays a check role and the guiding core tube (4) plays a guiding role; and the guiding core (5) is centered inside the catheter body (8);
a lateral liquid channel (22) is arranged between the front end of the guiding core (5) and the head end (1, 6) of the catheter so as to form the liquid turbulence within the cavity (17), reduce an injected liquid impacting on a patient's urethra from the lateral liquid channel (22), and make the patient comfortable; and
the catheter body (8) is more flexible than the head end (1, 6) of the catheter.

2. The catheter according to claim 1, wherein the cavity (17) comprises one or more cavities which are provided in series.

3. The catheter according to claim 1, wherein the head end (1, 6) of the catheter is directly connected with the guiding core (5), and an internal cross-section area of a front end of the catheter is the same as an inner hole area of the catheter.

4. The catheter according to claim 1, wherein the check means (2) or the guiding core head end (2) is directly arranged within the cavity (17) of the catheter and the front end of the guiding core (5) is in engagement with a hanging down column (16, 18) of the head end (1, 6) of the catheter.

5. The catheter according to claim 1, wherein at a tail end of the guiding core (5) and/or a guiding core tube (4), there is provided with a limiting means (13), so as to regulate a position of the guiding core (5) within the guiding core tube (4), so regulate a size of the lateral liquid channel.

6. The catheter according to claim 1, wherein at a tail end of the catheter, there is provided with a connecting means (10), and the connecting means (10) is provided with a conical hole (12) for discharging urine or for injecting lubrication.

7. The catheter according to claim 1, wherein the front end of the guiding core (5) is integrated with the head end (1, 6) of the catheter; or the front end of the guiding core (5) is matched with the head end (1, 6) of the catheter; or the check means (2, 51) or the guiding core head end (2) is directly arranged within the head end (1, 6) of the catheter.

8. The catheter according to claim 1, wherein a clamping means (51) of the front end of the guiding core (5) is matched with the head end (1, 6) of the catheter.

9. The catheter according to claim 1, wherein in view of a longitudinal direction of the catheter from its tail end to its head end (1, 6), the cavity (17) is provided to locate in front of any hole for discharging urine, making a cross-sectional area for injecting lubricant into the catheter is fully used to increase up to a whole inner cross section.

10. The catheter according to claim 1, wherein the cavity (17) is arranged to communicate with the lateral liquid channel (22), so as to form the liquid turbulence in a space of the cavity (17) before a liquid is injected out of the head end (1, 6).

11. The catheter according to claim 1, wherein a lubricant is injected through one of at least three ports (11, 12, 14) for injecting lubricant; while urine is discharged from one of at least two channels (12, 14) for discharging urine.

12. The catheter according to claim 1, wherein the head end (2) is operated to shrink into a guiding core tube (4) and move forward within the guiding core tube (4); after moving out of the guiding core tube (4), the head end (2) of the guiding core (5) expands outward, and the span of the expanded head end (2) of the guiding core (5) is greater than a size of the mouth of the cavity (17) at the head end (2) of the catheter.

13. The catheter according to claim 1, wherein the lateral liquid channel (22) communicates with a cylinder-shaped space between an outer cylinder surface of the guiding core (5) and an inner wall of the guiding core tube (4), and/or between an outer surface of the guiding core tube (4) and an inner wall of the catheter.

14. The catheter according to claim 6, wherein the connecting means (10) is further provided with a secondary hole for discharging urine, or the connecting means (10) is provided with a channel communicating with the hole (12) for discharging urine.

15. The catheter according to claim 1, wherein any accessory means within the guiding core tube (4) of the catheter is excluded so as to prevent from occupying any internal cross-sectional area of the catheter, prevent from sacrificing some volume for discharging urine by the catheter per minute; and prevent from increasing a resistance for injecting lubricant, while making a high efficiency for injecting lubricant, expanding a patient's urethra, reducing a friction between the catheter and the urethra, thus reducing any pain of the patient.

16. The catheter according to claim 1, wherein any over retracting displacement of the guiding core (5) within the guiding core tube (4) of the catheter is avoided, so that the guiding core (5) is prevented from accidently penetrating through a front hole (7) for discharge urine.

17. The catheter according to claim 1, wherein within the head end (2) of the catheter, there is provided a space (17) to communicate with the lateral liquid channel (22), so as to form the liquid turbulence in the space (17) before any liquid is injected out of the head end (2) of the catheter.

18. The catheter according to claim 1, wherein any additional attachment is prevented from providing on an inner surface of a tube wall of the catheter, so that in the catheter, an internal cross-sectional area for discharging urine becomes the same as the inner hole area of the catheter.
